(19) United States
(12) Patent Application Publication
(10) Pub. No.: US 2005/0209299 A1
(43) Pub. Date: Sep. 22, 2005

(54) Inhibition of mixed lineage kinases and uses therefor

(75) Inventor: Paul S. Shapiro, Baltimore, MD (US)

Correspondence Address: Robert T. Hrubiec;CEPHALON, INC., 145 Brandywine Parkway, West Chester, PA, 19380, US

(73) Assignee: University of Maryland, Baltimore, Baltimore, MD (US)

(21) Appl. No.: 11/081,929

(22) Filed: Mar. 15, 2005

Related U.S. Application Data
(60) Provisional application No. 60/553,497, filed on Mar. 16, 2004.

(51) Int. Cl. A61K 31/407

(52) U.S. Cl. 514/410

(57) ABSTRACT

Provided herein are methods of using an inhibitor of an MLK protein or polypeptide to inhibit cell proliferation in neoplastic cells, such as for example, a cancer. Such methods may be used to treat a cancer and further may be used in conjunction with administration of an anticancer drug at a reduced dosage to treat a cancer with a concomitant reduction in toxicity to an individual receiving the treatment. Also provided is a method to screen for inhibitory agents to inhibit an activity of a MLK protein or polypeptide and to inhibit cell proliferation of a neoplastic cell having the MLK activity.

### CROSS-REFERENCE TO RELATED APPLICATION

This non-provisional application claims benefit of provisional U.S. Ser. No. 60/553,497, filed Mar. 16, 2004, now abandoned.

### FEDERAL FUNDING LEGEND

This invention was produced in part using funds obtained through grant DAMD17-01-1-0548 from the Department of Defense. Consequently, the federal government has certain rights in this invention.

### BACKGROUND OF THE INVENTION

1. Field of the Invention

The present invention relates generally to the fields of medicine and cell biology. Specifically, the present invention relates to methods of preventing proliferation of cancer cells and of reducing toxicity of an anticancer compound during a cancer therapy. More specifically, the present invention relates to methods of inhibiting an activity of a multi-lineage kinase protein to prevent proliferation of cancer cells.

2. Description of the Related Art

Serine/threonine protein kinases, belonging to the mixed-lineage kinase (MLK) family, control the activity of mitogen-activated protein (MAP) kinases and are involved primarily in regulating tissue development and apoptotic responses (1). The MLK family includes MLK1-4, the dual leucine zipper-bearing kinase (DLK), and the leucine zipper kinase (LZK) (1-2). MLK proteins each contain highly conserved structural motifs that are important for protein interactions and signal transduction including a Src homology 3 (SH3) domain, a catalytic domain, two leucine zipper (LeuZip) motifs, and a Cdc42/Rac interactive binding (CRIB) motif (1). The regulation and function of the MLK1-4 subfamily has been best characterized for MLK2, which is localized to brain, skeletal muscle and testis, and MLK3, which is ubiquitously expressed (1). Currently, only partial sequence information is known for MLK1 and the physiological functions for MLK4 have not been defined.

MLK proteins are activated by upstream G-proteins including Cdc42 and Rac1, which recruit cytoplasmic MLK proteins to the plasma membrane by interacting with the CRIB motif (3). MLK protein activity may also be regulated through auto-inhibition involving intramolecular interactions between the SH3 domain and a single proline residue between the CRIB domain and the leucine zipper region (4). Recently, the stability of MLK3 has been linked to the ability for MLK3 to interact with heat shock protein-90 (HSP90), where recent data suggests that geldanamycin inhibition of HSF90 results in decreased MLK3 stability and expression (5).

The functional role of MLK proteins in activating the stress-activated protein kinases and apoptotic pathways in neuronal cells has been well characterized (6-8). MLK3 is a potent activator of the c-Jun N-terminal kinase (JNK) MAP kinases by directly phosphorylating and activating the JNK kinases, MKK4 and MKK7 (9-10). MLK3 may also activate the p38 MAP kinases through direct phosphorylation of MKK3 and MKK6, which are the primary p38 MAP kinase activators (10). MLK proteins have been proposed to play a critical role in the progression of neurodegenerative diseases, such as Parkinson's, Huntington's, and Alzheimer's diseases, through a mechanism involving MLK induced JNK activation, cyctochrome c release, and caspase activation of apoptotic pathways (6, 8). Thus, several small molecule inhibitors of MLK activity belonging to the indolocarbazole family, which include CEP-1347 (KT7515) and CEP-11004 (KT-8138), are being tested in clinical trials and may prove beneficial in preventing premature neuronal cell death (6-8, 11-12).

In non-neuronal cells MLK proteins may also function in promoting cell proliferation. For example, overexpression of MLK3 has been reported to induce NIH 3T3 cell transformation and growth on soft agar through a mechanism involving MEK1 and ERK activation (13). Similarly, growth factors may utilize MLK3 to activate B-Raf and the ERK pathway in proliferating tumor cells (14). MLK3 activity may regulate cell proliferation by affecting centrosome organization and microtubule stability during mitosis through a mechanism that is JNK-independent (15). Additional evidence suggests that MLK3 may promote cell transformation by mediating morphological changes in cells expressing an activated Rac1 mutant (16). Recently, overexpression of a MLK-like protein, MLTK, was shown to be sufficient to induce cell transformation in a nude mouse model (17). Also, a recent report indicates that MLK3 is upregulated in breast cancer cell lines (5). Thus, ample evidence links MLK proteins with promoting cell proliferation.

There is a need in the art for methods of regulating proliferation of transformed cells to treat a pathophysiological state or condition caused thereby. The prior art is deficient in the lack of methods of inhibiting proliferation of cancer cells. Specifically, the prior art lacks methods of inhibiting an MLK protein to arrest cancer cell proliferation. The present invention fulfills this long-standing need and desire in the art.

### SUMMARY OF THE INVENTION

The present invention is directed to a method of inhibiting proliferation of a neoplastic cell. The method comprises contacting the neoplastic cell with a compound that selectively inhibits an activity of a mixed-lineage kinase (MLK). Inhibition of a MLK activity thereby inhibits proliferation of the neoplastic cell.

The present invention is directed to a related method of inhibiting proliferation of a neoplastic cell. The neoplastic cell is contacted with a compound that selectively inhibits an activity of mixed lineage kinase (MLK). The mixed lineage kinase has a sequence selected from the group consisting of SEQ ID NO:1, SEQ ID NO:2, SEQ ID NO:3, and SEQ ID NO:4, where the inhibition of the MLK activity inhibits proliferation of the neoplasm.

The present invention also is directed to a method of treating a cancer in a subject. The method comprises administering an inhibitor that selectively binds to a mixed-lineage kinase (MLK) or polypeptide thereof and inhibiting an activity of the MLK or MLK polypeptide upon binding the inhibitor to arrest proliferation of cancer cells. Arresting proliferation of the cancer cells treats the cancer. The method may comprise a further step of administering an anticancer drug to the individual.

The present invention is directed to a related method of reducing toxicity of a cancer therapy in an individual in need thereof. The method comprises co-administering to the individual an inhibitor that selectively binds to a mixed-lineage kinase (MLK) or polypeptide thereof and an anticancer drug. The dosage of the anticancer drug administered with the inhibitor is lower than a dosage required when the anticancer drug is administered singly. Toxicity of the cancer therapy to the individual is thereby reduced.

The present invention is directed further to a method of screening for a compound to inhibit mixed lineage kinase (MLK) activity and to arrest proliferation of a neoplastic cell. The level of an activity of a MLK protein or of a polypeptide thereof or polypeptide fragment having the MLK activity is measured in the presence or absence of the compound. A decrease in the level of MLK activity in the presence of the compound compared with the level of MLK activity in the absence of the compound is indicative that the compound has an ability to inhibit MLK activity. A culture of neoplastic cells having an activated MLK activity is contacted with the compound having an ability to inhibit the MLK activity. The amount of cell proliferation of the neoplastic cells in the presence of the inhibitory compound with the amount of cell proliferation of the neoplastic cells in the absence of the inhibitory compound is compared. A decrease in cell proliferation in the presence of the inhibitory compound compared to cell proliferation in the absence of the inhibitory compound is indicative that the inhibitory compound has the ability to prevent cell proliferation.

The present invention is directed further still to compounds identified by the screening methods described herein. These compounds inhibit an activity of a mixed lineage kinase or polypeptide thereof and arrest proliferation of neoplastic cells. These compounds may be used in any of the methods of inhibiting cell proliferation of a neoplastic cell, of treating a cancer or of reducing toxicity of an anticancer drug described in the present invention.

Other and further objects, features, and advantages will be apparent from the following description of the presently preferred embodiments of the invention, which are given for the purpose of disclosure.

### BRIEF DESCRIPTIONS OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1B demonstrate that inhibition of MLK activity blocks cell proliferation. FIG. 1A shows HeLa or NIH 3T3 cells grown in the absence (closed squares or circles) or presence (open squares or circles) of CEP-11004 (500 nM) for up to 72 hours. The number of cells was counted at time 0 and after 24, 48, or 72 hours in each condition. FIG. 1B shows trypsinized A549 cells (1000 per treatment) plated in the presence of varying doses of CEP-11004. After 14 days, the resulting colonies were stained with crystal violet, and counted. Similar results were obtained with HeLa and SUM152 breast cancer cells.

FIGS. 2A-2D demonstrate that CEP-11004 causes G2/M-phase arrest in transformed cells. HeLa (FIG. 2A), HEK293 (FIG. 2B), NIH3T3 (FIG. 2C), or MRC5 (FIG. 2D) cells were treated in the absence (control) or presence of CEP-11004 (500 nM) for 15 hours and 2N or 4N DNA content in cells was measured by FACS analysis following propidium iodide staining.

FIGS. 3A-3D demonstrate that CEP-11004 treatment of synchronized cells during late S-phase/early G2-phase causes G2/M-phase arrest. HeLa cells were synchronized at the G1/S-phase boundary by double thymidine block, released back into the cell cycle, and treated 5 hours after release in the absence (FIG. 3A) or presence (FIG. 3B) of CEP-11004 (500 nM). Cells were collected by trypsinization at indicated various times after G1/S release, the DNA was stained with propidium iodide, and the percentage of cells in G1, S, or G2/M-phase was determined by FACS. In FIG. 3C HeLa cells were synchronized as in FIG. 3A, released into the cell cycle, and treated in the absence or presence of CEP-11004 (500 nM). Lysates collected at various times were immunoblotted for cyclin B1 (top panel) or total MKK2 as a protein loading control (lower panel). In FIG. 3D histone kinase activity in synchronized cells was determined in untreated (closed circles) or CEP-11004 treated (open circles) cells following immunoprecipitation of Cdc2. Data shows the average and standard error from 3 independent experiments.

FIGS. 4A-4E demonstrate that CEP-11004 targets MLK3 but not DLK or MKK1 activity. In FIG. 4A HeLa cells were transfected with HA-tagged MLK3 wild type (WT) or a catalytically inactive MLK3 (KR) mutant. The MLK3 wild type transfected cells were incubated with or without CEP-11004 (500 nM) for 4 hours prior to harvesting. Wild type and inactive MLK3 were immunoprecipitated with a MLK3 antibody and incubated with MBP as a substrate in an in vitro kinase assay. The graph shows the relative amount of phosphate incorporation into MBP under each condition and the immunoblot shows the amount of the MLK3 proteins in the immunoprecipitates. In FIG. 4B HeLa cells were transfected with HA-tagged JNK1 plus MLK3 wild type or Flag-tagged DLK and then treated in the absence or presence of CEP-11004 (500 nM) for 4 hours prior to harvesting. Immunoblots for active JNK (pJNK), HA-JNK1, Flag-DLK, or HA-MLK3 are shown in the top to bottom panels, respectively. In FIG. 4C HeLa cells were transfected with wild type (WT) MLK3, inactive (KR) MLK3, or a constitutively active MKK1 mutant (CA) and then treated with or without CEP-11004 (500 nM). The relative level of active ERK was determined by immunoblotting with a phospho-specific ERK1/2 (ppERK) antibody (upper panel). Protein loading was monitored by immunoblotting for α-tubulin (lower panel). In FIG. 4D HeLa cells were co-transfected with HA-JNK1 and GFP tagged MLK2 or HA-MLK3. Transfected cells were then treated for 1 hour with various concentrations of CEP-11004 and JNK activity was determined by immunoblotting with a phospho-specific JNK1/2 antibody (pJNK, upper panel). The expression of HA-JNK1, GFP-MLK2, or HA-MLK3 is shown in the lower panels. The upper and lower arrows in the HA-MLK3 blot indicate phosphorylated and de-phosphorylated forms of MLK3, respectively. FIG. 4E is a graph of the relative JNK activity as quantified by measuring the ratio of pJNK to total JNK by densitometry in cells expressing MLK2 (closed squares) or MLK3 (open squares).

FIGS. 5A-5B demonstrate that CEP-11004 causes cell cycle arrest in pro-metaphase. HeLa cells grown on coverslips were synchronized at the G1/S boundary with excess thymidine and released back into the cell cycle. At 5 hours after release, cells were treated in the absence or presence of CEP-11004 (500 nM) for an additional 4 or 6 hours (9 or 11 hours cumulative time after G1/S release). In FIG. 5A the percentage of cells in prophase (P), pro-metaphase (PM), metaphase (M), or anaphase/telophase (AT) was determined by DAPI staining of chromosomes in control (open bars) or CEP-11004 treated (closed bars) cells. The left and right graphs represent the percentage of mitotic cells 9 and 11 hours, respectively, after G1/S-phase release. Data represent the average and standard error from 3 independent experiments. In FIG. 5B the number of cells in pro-metaphase were counted at 11 hours after G1/S-phase release following treatment with increasing concentrations of CEP-11004 (added at 5 hours after G1/S-phase release). 300-350 cells were counted at each concentration.

FIGS. 6A-6C demonstrate that exogenous MLK3 inhibits CEP-11004-induced mitotic arrest. HeLa cells were co-transfected with pEGFP in the presence or absence (mock) of HA-tagged MLK3 wild type and then synchronized by double thymidine block. At 5 hours after G1/S release, the cells were treated with various concentrations of CEP-11004 (75, 100, 200 nM) for 8 hours and then harvested (13 hours post G1/S release). FIG. 6A shows immunoblots for cyclin B1 (top panel), MLK3 (middle panel), and β-actin (lower panel) as a protein loading control. In FIG. 6B synchronized HeLa cells transfected with pEGFP plus or minus MLK3 were grown on coverslips, treated with or without various concentrations of CEP-11004, fixed at 13 hours after release from G1/S, and stained with DAPI. The number of mitotic cells (prophase, pro-metaphase, metaphase, and anaphase/telophase cells) was determined by DAPI staining in GFP positive cells (closed bars) or GFP positive cells that co-express MLK3 (open bars). FIG. 6C shows relative Cdc2 kinase activity in lysates collected 13 hours after G1/S release in cells that were transfected with pEGFP alone or pEGFP and MLK3. Radiolabeled phosphate incorporation into histone substrate (upper panel) and the immunoblot for Cdc2 in the immunoprecipitates (lower panel) are shown. IgG_{(L)} indicates the antibody light chain.

FIGS. 7A-7B demonstrate that CEP-11004 delays nuclear histone H3 phosphorylation in early prophase. HeLa cells grown on coverslips were synchronized at G1/S with excess thymidine and released back into the cell cycle. At 5 hours after release, cells were incubated in the absence or presence of CEP-11004 (500 nM). After an additional 4 hours incubation (9 hours after G1/S release), cells were fixed and co-immunostained for cyclin B1 or phosphorylated histone H3 (pH3) in the top and middle panels, respectively (FIG. 7A). MLK3 and cyclin B1 expression are shown in the top and middle panels, respectively (FIG. 7B). Mitotic cells (indicated by arrows) were determined by the presence of nuclear cyclin B1 and/or pH3 staining. Cells in FIGS. 7A-7B were counterstained with DAPI (lower panels).

FIGS. 8A-8B demonstrate that CEP-11004 inhibits histone H3 phosphorylation prior to nuclear envelope breakdown. HeLa cells were prepared for immunostaining as described in FIGS. 5A-5B. Cells were stained for pH3 (top panels) and nucleoporin (p62, middle panels) as a marker of the nuclear envelope. Cells in prophase (pre nuclear envelope breakdown) (FIG. 8A) or pro-metaphase (post nuclear envelope breakdown) (FIG. 8B) that had been treated in the absence or presence of CEP-11004 were identified. Mitotic cells, as indicated by the arrows, were identified by DAPI staining (bottom panels) of condensed chromosomes. Adjacent interphase cells are shown for comparison.

FIGS. 9A-9B demonstrate that CEP-11004 induces aberrant mitotic spindles. HeLa cells grown on coverslips were treated in the absence or presence of CEP-11004 as described FIG. 5. In FIG. 9A cells were collected at 9 hours after release from G1/S block and the organization of the mitotic spindle was evaluated by immunostaining for α or γ-tubulin to identify microtubules and the spindle poles. Cells were counterstained with DAPI to identify mitotic chromosomes. The merged images shows α-tubulin in green, γ-tubulin in red, and DAPI in blue pseudocolor. In FIG. 9B the percentage of the mitotic cells with aberrant spindles in control or CEP-11004 treated cells was quantified in 3 separate experiments. At least 300 mitotic cells were counted in each experiment under each condition.

### DETAILED DESCRIPTION OF THE INVENTION

In one embodiment of the present invention there is provided a method of inhibiting proliferation of a neoplastic cell, comprising contacting the neoplastic cell with a compound that selectively inhibits an activity of a mixed-lineage kinase (MLK), thereby inhibiting proliferation of the neoplastic cell. In all aspects of this embodiment, the neoplastic cell may comprise a cancer. Representative examples of a cancer are a breast cancer, a lung cancer, a cervical cancer, a pancreatic cancer, a bladder cancer, a colon cancer, or any cancer having a Ras mutation. In all aspects of this embodiment the compound may be an indolocarbazole molecule. Representative examples are CEP-11004 or CEP-1347.

In one aspect the mixed lineage kinase may be a MLK1 polypeptide having the sequence shown in SEQ ID NO:3. In another aspect the mixed lineage kinase may be a MLK2 protein having the sequence shown in SEQ ID NO:4. In yet another aspect the mixed lineage kinase may be a MLK3 protein having the sequence shown in SEQ ID NO:1 or in SEQ ID NO:2.

In a related embodiment there is provided a method of inhibiting proliferation of a neoplastic cell, comprising contacting the neoplastic cell with a compound that selectively inhibits an activity of mixed lineage kinase (MLK) having a sequence selected from the group consisting of SEQ ID NO:1, SEQ ID NO:2, SEQ ID NO:3, and SEQ ID NO:4, where the inhibition of the MLK activity inhibits proliferation of the neoplasm. In all aspects of this embodiment the neoplastic cell may comprise a cancer as described supra. Additionally, in all aspects the inhibitor is as described supra.

In another embodiment of the present invention there is provided a method of treating a cancer in a subject, comprising administering an inhibitor that selectively binds to a mixed-lineage kinase (MLK) or polypeptide thereof; inhibiting an activity of the MLK or MLK polypeptide upon binding said inhibitor such that proliferation of cancer cells is arrested, thereby treating the cancer in the subject.

Further to this embodiment the method may comprise administering an anticancer drug to the subject. In aspects of this embodiment, the anticancer drug may be administered concurrently or sequentially with the MLK inhibitor. In another aspect of this embodiment a dosage of the anticancer drug is lower than a dosage required when the anticancer drug is administered singly, thereby reducing toxicity of the anticancer drug to the individual. Examples of anticancer drugs are cisplatin, oxaliplatin, carboplatin, doxorubicin, a camptothecin, paclitaxel, methotrexate, vinblastine, etoposide, docetaxel hydroxyurea, celecoxib, fluorouracil, busulfan, imatinib mesylate, alembuzumab, aldesleukin, and cyclophosphamide. Additionally, in all aspects the types of cancer, MLK inhibitors and the MLK proteins and polypeptides thereof are as described supra.

In a related embodiment the present invention provides a method of reducing toxicity of a cancer therapy in an individual in need thereof, comprising administering to the individual an inhibitor that selectively binds to a mixed-lineage kinase (MLK) or polypeptide thereof and an anticancer drug, wherein a dosage of the anticancer drug administered with the inhibitor is lower than a dosage required when the anticancer drug is administered singly, thereby reducing toxicity of the cancer therapy to the individual. In aspects of this embodiment, the anticancer drug may be administered concurrently or sequentially with the MLK inhibitor. In all aspects of this embodiment the MLK inhibitors, the MLK proteins and polypeptides, the anticancer drugs and the types of cancer are as described supra.

In yet another embodiment of the present invention, there is provided a method of screening for a compound to inhibit mixed lineage kinase (MLK) activity and to arrest proliferation of a neoplastic cell, comprising a) measuring the level of an activity of a MLK protein or of a polypeptide thereof or polypeptide fragment having said MLK activity in the presence or absence of the compound; b) comparing the level of MLK activity in the presence of the compound with the level of MLK activity in the absence of the compound, wherein a decrease in MLK activity in the presence of the compound is indicative that the compound has an ability to inhibit MLK activity; c) contacting a culture of the neoplastic cells having an activated MLK activity with the compound having an ability to inhibit the MLK activity; and d) comparing the amount of cell proliferation of the neoplastic cells in the presence of the inhibitory compound with the amount of cell proliferation of the neoplastic cells in the absence of the inhibitory compound, wherein a decrease in cell proliferation in the presence of the compound compared to cell proliferation in the absence of the compound is indicative that the inhibitory compound has the ability to prevent cell proliferation.

In all aspects of this embodiment, the mixed lineage kinase is a MLK2 protein or a MLK3 protein or a MLK1 polypeptide or a polypeptide fragment having an MLK activity. The MLK1 polypeptide may have the sequence shown in SEQ ID NO:3. The MLK2 polypeptide may have the sequence shown in SEQ ID NO:4. The MLK3 polypeptide may have the sequence shown in SEQ ID NO:1 or in SEQ ID NO:2. The MLK polypeptide fragment having the MLK activity may comprise about 700, 650, 600, 550, 500, 450, 400, 350, 300, 250, 200, 150, or 100 continuous amino acids of the MLK2 or MLK3 proteins or of the MLK1 polypeptide. In all aspects the neoplastic cell and the types of cancer are as described supra.

In a related embodiment there is provided a compound identified by the methods of screening for an inhibitor of a mixed lineage kinase activity and of cell proliferation of a neoplastic cell.

The following abbreviations are used herein. Mixed-lineage kinase, MLK; Mitogen-Activated Protein, MAP; Extracellular signal-Regulated Kinase, ERK; c-Jun NH₂-terminal Kinase, JNK; Dual Leucine Zipper-Bearing Kinase, DLK; Cdc42/Rac Interactive Binding, CRIB; CEP-11004, [3,9-bis-[(isopropylthio)methyl]-(8R*,9S*, 11S*)-(−)-9-hydroxy-9-methoxycarbonyl-8-methyl-2,3,9,10-tetrahydro-8,11-epoxy-1H,8H,11H-2,7b,11a-triazadibenzo(a,g)cycloocta(cde)trinden-1-one]; CEP-1347 [3,9-bis[(ethylthio)methyl]-(8R*,9S*,11-S*)-(−)-9-hydroxy-9-methoxycarbonyl-8-methyl 2,3,9,10-tetrahydro-8,11-epoxy-1H,8H, 11H-2,7b,11a-triazadibenzo(a,g)cycloocta(cde)trinden-1-one].

As used herein, the term, “a” or “an” may mean one or more. As used herein in the claim(s), when used in conjunction with the word “comprising”, the words “a” or “an” may mean one or more than one. As used herein “another” may mean at least a second or more.

As used herein, the term “contacting” refers to any suitable method of bringing an inhibitory agent into contact with a MLK protein or polypeptide or fragment thereof, as described, or a cell comprising the same. In vitro or ex vivo this is achieved by exposing the MLK protein or polypeptide or fragment thereof or cells comprising the same to the inhibitory agent in a suitable medium. For in vivo applications, any known method of administration is suitable as described herein.

As used herein, the term “neoplasm” refers to a mass of tissue or cells characterized by, inter alia, abnormal cell proliferation. The abnormal cell proliferation results in growth of these tissues or cells that exceeds and is uncoordinated with that of the normal tissues or cells and persists in the same excessive manner after the stimuli which evoked the change ceases or is removed. Neoplastic tissues or cells show a lack of structural organization and coordination relative to normal tissues or cells which usually results in a mass of tissues or cells which can be either benign or malignant. As would be apparent to one of ordinary skill in the art, the term “cancer” refers to a malignant neoplasm.

As used herein, the term “transformed” or the phrase “transformed cell” refers to a cell that exhibits neoplastic growth.

As used herein, the term “treating” or the phrase “treating a cancer” or “treating a neoplasm” includes, but is not limited to, halting the growth of the neoplasm or cancer, killing the neoplasm or cancer, or reducing the size of the neoplasm or cancer. Halting the growth refers to halting any increase in the size or the number of or size of the neoplastic or cancer cells or to halting the division of the neoplasm or the cancer cells. Reducing the size refers to reducing the size of the neoplasm or the cancer or the number of or size of the neoplastic or cancer cells.

As used herein, the term “subject” refers to any target of the treatment.

As used herein, the term “MLK inhibitor” means a molecular entity of natural, semi-synthetic or synthetic origin that blocks, stops, inhibits, and/or suppresses an activity of a mixed lineage kinase (MLK) polypeptide, including, but not limited to, a MLK1, a MLK2 and/or a MLK3 polypeptide.

As used herein, the term “MLK polypeptide” is used interchangeably with “MLK protein.” The experiments described herein focused on the MLK3 isoform for the following reasons. First, MLK3 is ubiquitously expressed and the best characterized of the MLK proteins. Second, although MLK2 shares more than 70% sequence identify in the kinase catalytic domain and other regions with MLK3, the expression of MLK2 polypeptide is restricted to brain and muscle tissue. Third, the full sequence of MLK1 has yet to be identified. However, it is contemplated that the inhibition of the human MLK3 (SEQ ID NO:1) polypeptide is a non-limiting exemplary embodiment and that the inhibition of a MLK3 polypeptide in a different mammal, such as a mouse (SEQ ID NO:2), and/or the human MLK2 polypeptide (SEQ ID NO:4) and/or the human MLK1 (SEQ ID NO:3) polypeptide are contemplated as within the spirit and scope of the invention.

The present invention discloses that MLK proteins or polypeptides serve as an important regulator of microtubule formation during mitosis in transformed cells exhibiting neoplastic growth. Subsequently, inhibition of MLK proteins or polypeptides blocks mitotic progression in a pro-metaphase like arrest due to the inability to properly form a mitotic spindle necessary for metaphase to anaphase transitions. Inhibition of MLK proteins may preferentially inhibit mitotic transitions and cell proliferation of transformed cells, but not in normal cells. The inherent abnormalities of cell cycle regulation in the transformed cells may make these cells more sensitive to undergo cell cycle arrest and apoptosis in response to MLK inhibitors. Thus MLK proteins or polypeptides serve as a unique target for treatment of cancer cell proliferation without affecting normal cell function.

Provided herein are methods that utilize MLK polypeptides as a unique target for treating a neoplasm, e.g., but not limited to, a cancer, such that neoplastic cell proliferation is arrested, stopped, blocked, or ceases to occur without affecting normal untransformed cell function. Accordingly, the present invention is drawn to MLK inhibitors that bind specific polypeptide sequences of an MLK protein and inhibit or interfere with an activity thereof to affect cancer cell proliferation. The MLK inhibitor may block, stop, inhibit, and/or suppress an activity of an MLK polypeptide, that is, but not limited to, a full-length polypeptide such as SEQ ID NOS: 1, 2 and/or 4 or a peptide fragment thereof, such as SEQ ID NO:3. A polypeptide fragment also may comprise about 700, 650, 600, 550, 500, 450, 400, 350, 300, 250, 200, 150, or 100 continuous amino acids of the MLK polypeptide, including a MLK1 polypeptide of SEQ ID NO:3, a MLK2 polypeptide of SEQ ID NO: 4 or a MLK3 polypeptide of SEQ ID NOS:1 or 2.

The MLK inhibitor may be an indolocarbazole compound or a derivative or analog thereof or other molecule effective to interfere with an activity of an MLK protein or polypeptide, such as a kinase activity. Representative inhibitors are CEP-11004 or CEP-1347. It is contemplated that CEP-11004 selectively binds to MLK3. It also is contemplated that potential compounds may be screened for their ability to inhibit an activity of a MLK protein or polypeptide and to inhibit cell proliferation of a transformed cell. For example, a cancer cell culture having activated MLK activity is contacted with a potential MLK inhibitor. A decrease in cell proliferation, as compared to control, may be determined by standard assays, such as trypan blue exclusion or a colony formation assay. Compounds effective to inhibit an activity of a MLK protein or polypeptide and to inhibit cell proliferation may be screened further by FACS analysis of DNA content to characterize the nature of the decrease in cell proliferation.

Alternatively, potential inhibitors of MLKs may be screened initially by contacting the potential inhibitor with one or more MLK proteins or polypeptides described herein in the presence of a known substrate for an MLK-associated activity. For example, the MLK-associated activity may be assayed in the presence of ATP and a substrate phosphorylated via MLK activity and in the presence or absence of the potential MLK inhibitor. A decrease in MLK activity in the presence of the potential inhibitor compared to activity in the absence of the potential inhibitor is indicative that it has an ability to inhibit an MLK activity. Such enzyme assays are known and standard in the art. The MLK polypeptide may be any of MLK1-4 or may be a fragment of thereof, as described, provided that the polypeptide fragments retain at least one activity associated with the MLK1-4 polypeptide. Subsequently, any potential inhibitor of the MLK-associated activity may be used in the cell proliferation assays as described.

Potential inhibitors of cell proliferation of cancer cells may be natural, semi-synthetic or synthetic compounds known to inhibit or interfere with an activity of an MLK protein or may be compounds screened from chemical libraries or may be a synthetic derivative or analog compound having a structure similar to a known inhibitor. Inhibitors of MLKs identified by the methods described herein can block proliferation of cancer cells without affecting normal cell proliferation. Such inhibitors may be used to inhibit proliferation of neoplastic cells, to treat a cancer or to reduce the toxicity of a cancer drug to normal cells.

The MLK inhibitors may be used to treat any subject, preferably a mammal, more preferably a human, having a pathophysiological condition characterized by the presence of transformed cells, e.g., a neoplasm, such as, but not limited, to a cancer. For example, a cancer may be a breast cancer, a lung cancer, a cervical cancer, a pancreatic cancer, a bladder cancer, a colon cancer, or another cancer having a Ras mutation. Administration of an MLK inhibitor to a subject results in growth arrest of cancer cells without affecting the growth of a normal cell. Thus, cell proliferation is inhibited and a therapeutic effect, up to and including killing the cancer, is achieved thereby treating the cancer. It is contemplated that the MLK inhibitors of the present invention may be used to inhibit proliferation of non-malignant neoplastic diseases and disorders.

An anticancer drug may be administered concurrently or sequentially with the MLK inhibitor. The effect of co-administration with an MLK inhibitor is to lower the dosage of the anticancer drug normally required that is known to have at least a minimal pharmacological or therapeutic effect against a cancer or cancer cell, for example, the dosage required to eliminate a cancer cell. Concomitantly, toxicity of the anticancer drug to normal cells, tissues and organs is reduced without reducing, ameliorating, eliminating or otherwise interfering with any cytotoxic, cytostatic, apoptotic or other killing or inhibitory therapeutic effect of the drug on the cancer cells.

MLK inhibitors and anticancer drugs can be administered independently, either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of MLK inhibitors may comprise conventional non-toxic, physiologically or pharmaceutically acceptable carriers or vehicles suitable for the method of administration.

The MLK inhibitors and anticancer drugs or pharmaceutical compositions thereof may be administered independently one or more times to achieve, maintain or improve upon a therapeutic effect. It is well within the skill of an artisan to determine dosage or whether a suitable dosage of either or both of the MLK inhibitor and anticancer drug comprises a single administered dose or multiple administered doses. An appropriate dosage depends on the subject's health, the progression or remission of the cancer, the route of administration and the formulation used.

The following examples are given for the purpose of illustrating various embodiments of the invention and are not meant to limit the present invention in any fashion.

### EXAMPLE 1

Cell Culture and Reagents

HeLa, NIH 3T3, HEK293, A549, or MRC-5 cells were grown in a DMEM supplemented with 10% fetal bovine serum (FBS) and penicillin (100 U/ml) and streptomycin (100 μg/ml). Estrogen receptor (ER) negative breast cancer cells, SUM-159, were obtained from the University of Michigan Human Breast Cancer Cell Lines (SUM-Lines) and grown in DMEM+10% FBS. In some experiments, cells were transfected with cDNA (1 μg) for pEGFP (BD Biosciences/Clontech, Palo Alto, Calif.), hemagglutinin (HA)-tagged MLK3, kinase dead mutant MLK3 (MLK3 K144R), GFP-tagged MLK2 (kindly provided by Dr. Donna Dorow), Flag-tagged DLK, or HA-tagged JNK1 using Lipofectamine® (Invitrogen, Carlsbad, Calif.). Transfected cells were harvested 16-24 hours after transfection.

Antibodies specific for MLK3 (C-20), JNK (C-17), phospho-JNK1/2 (G-7), Cyclin B1 (GNS1), MKK2 (C-16), and Cdc2 (#17) were purchased from Santa Cruz Biotech (Santa Cruz, Calif.). Antibodies for p62 nucleoporin (N43620) and phospho-specific histone H3 (serine 10) (Cat# 07-081) were purchased from BD Biosciences (Palo Alto, Calif.) and Upstate Biotechnology (Charlottesville, Va.), respectively. CEP-11004 (kindly provided by Cephalon Inc. West Chester, Pa.) was reconstituted at a stock concentration of 0.4 mM in DMSO.

### EXAMPLE 2

Cell Synchronization

In some experiments, cells were synchronized by double thymidine block as previously described (18-19). Briefly, cells were incubated for 16 hours with 2 mM thymidine in 10% FBS containing DMEM. The excess thymidine was washed off with Hanks Buffered Saline Solution (HBSS) and cells were released back into the cell cycle with complete medium. After 8 hours, cells were treated a second time with 2 mM thymidine in 10% FBS containing DMEM for another 16 hours to arrest cells at the G1/S-phase boundary. Synchronized cells were then washed with HBSS and released back into the cell cycle and harvested at various time points after G1/S release for analysis. Cell cycle analysis was done by fluorescence activated cell sorting (FACS) as described below. In some experiments, CEP-11004 was added to the synchronized cells 5 hours after release from G1/S block, which corresponded to late S-phase or early G2-phase.

### EXAMPLE 3

Cell Proliferation Assays

Following treatments cells were counted on a hemocytometer following trypsinization and staining with trypan blue (Sigma). Greater than 95% of the cells excluded trypan blue dye. Cell proliferation also was measured using a colony formation assay. Growing cells were trypsinized and replated (1000 cells per 100 mm dish) in the presence or absence of various concentrations of CEP-11004. Following incubation for 8-14 days, cells were fixed for 10 minutes in 4% paraformaldehyde and stained with 0.2% crystal violet (in 20% methanol) for 1-2 minutes. Cells were washed several times with distilled water and colonies formed (at least 40 cells/per colony) were counted.

### EXAMPLE 4

Immunoblotting and Immunoprecipitation

For protein analysis, cells were washed twice with cold phosphate buffered saline (PBS), lysed with 300 μl of tissue lysis buffer (TLB) (20 mM Tris-base, pH 7.4, 137 mM NaCl, 2 mM EDTA, 1% Triton X-100, 25 mM β-glycerophosphate, 2 mM sodium pyrophosphate, 10% glycerol, 1 mM sodium orthovanadate, 1 mM phenylmethysulfonyl fluoride (PMSF), and 1 mM benzamidine) and centrifuged at 20,000×g to clarify lysates. Approximately 20 μg of total protein was separated by SDS-PAGE. Proteins were transferred to PVDF membrane (Perkin Elmer Life Sciences; Boston, Mass.), blocked for 1-2 hours with 5% nonfat dry milk in Tris-buffered saline (TBS) (50 mM Tris-base, pH 7.4, 0.15 M NaCl, and 0.1% Tween-20) and incubated with the primary antibodies in TBS containing 1% BSA solution for 1 to 16 hours. Membranes were washed several times in TBS-Tween solution and incubated with HRP conjugated anti-mouse or anti-rabbit antibodies (0.1 μg/ml). Immunoreactivity was detected by enhanced chemiluminescence (ECL; Amersham, Buckinghamshire, England).

In some experiments, proteins were immunoprecipitated with 1 μg/ml of primary antibodies incubated with cell lysate for 4-16 hours at 4° C. Protein A or G sepharose (Amersham/Pharmacia, Uppsala, Sweden)) was added for an additional 4 hours and the immune complexes were washed twice with kinase buffer (KB, 25 mM Hepes, pH 7.4, 25 mM MgCl₂, and 1 mM DTT). The proteins in the immune-complex were separated by SDS-PAGE and analyzed by immunoblotting.

### EXAMPLE 5

Immunofluorescence

Cells grown on round glass coverslips in 6-centimeter plates were synchronized at the G1/S-phase boundary using excess thymidine as described in Example 4. At the indicated time after release back into the cell cycle, coverslips were fixed with 4% paraformaldehyde for 8 minutes and permeabilized with 0.1% Triton X-100 in PBS for 2 minutes. Cell staining patterns were confirmed in cells that were fixed and permeabilized with cold methanol for 10 minutes. Cells were stained with antibodies against MLK3, α-tubulin, cyclin B1, phospho-specific histone H3, or p62 nucleoporin followed by incubation with fluorescein or Texas Red conjugated secondary antibodies and counterstaining for cellular DNA with 4′,6-diamidino-2-phenylindole (DAPI, 0.2 μg/ml in PBS). Cells were identified using Nikon E800 Epi-fluorescence microscope (Image Systems, Columbia, Md.) and captured with a Hamamatsu CCD camera. Cell images were processed using IPlab software (Scanalytics, Fairfax, Va.).

### EXAMPLE 6

MLK3 Kinase Assays

Wild type MLK3 or catalytically inactive MLK3KR (K144R) were immunoprecipitated from transfected cells treated with or without CEP-11004 and the immune-complex was washed with cold KB supplemented with 0.2 mM sodium orthovanadate. Immunoprecipitated MLK3 was incubated for 45 min at 30° C. with kinase buffer containing 10 μCi γ-³²P ATP, 20 μM cold ATP, and 0.5 μg myelin basic protein (MBP) as a substrate. The reaction was quenched with 2×SDS sample buffer, separated by SDS-PAGE and transferred to PVDF membranes (Perkin Elmer, Boston, Mass.). Radioactive phosphate incorporation into MBP and MLK3 were analyzed by phosphorimager (Amersham Biosciences/Molecular Dynamics) and immunoblotting, respectively.

### EXAMPLE 7

FACS Analysis.

Synchronized cells were trypsinized, washed with PBS, fixed with 3 ml of cold (−20° C.) 70% ethanol, and stored at 4° C. overnight. Cells then were incubated with 100 μg/ml propidium iodide (Sigma) dissolved in 0.2 M Tris, pH 7.5, 20 mM EDTA, 1 mg/ml RNase A (Sigma) for 1 hour at room temperature and diluted with an equal volume of PBS. DNA content was measured by flow cytometry (FACScan Analyzer, BD Biosciences) and was analyzed using the Sync Wizard Model, ModFit LT software (BD Biosciences). To determine the percentage of G1, S and G2/M-phase cells, the settings for 2N and 4N peaks were defined within each experiment from the G1/S arrested cells and applied to all samples within a given experiment.

### EXAMPLE 8

Cdc2 Kinase Activity

Cdc2 was immunoprecipitated from cell lysates by incubating with Cdc2 antibody (0.5 μg) for 2 hours on ice. The lysates then were incubated with protein G-sepharose with mixing at 4° C. for an additional 2 hours. The immune-complex was washed with cold kinase buffer and incubated for 30 min at 30° C. with kinase buffer containing 10 μCi γ-³²P ATP, 20 μM cold ATP, and 2.5 μg histone (Type III-SS, Sigma) as a substrate. The reactions were stopped with SDS-PAGE sample buffer and the proteins were resolved by SDS-PAGE. Incorporation of ³²P into histone was determined by phosphorimager analysis.

### EXAMPLE 9

Mitotic Index Assay

Cells grown on coverslips were synchronized at the G1/S-phase boundary as described in Examples 4-5. In some cases, MLK3 was co-expressed with the pEGFP vector in order to identify transfected cells. At 5 hours after release from thymidine-induced G1/S-phase block, cells were incubated in the presence or absence of varying concentrations of CEP-11004. At varying times after release, coverslips were fixed and cellular DNA was stained with DAPI. GFP-positive mitotic cells in prophase, prometaphase, metaphase, anaphase, or telophase were identified, counted, and expressed as a fraction of the total cells counted to determine the mitotic index. Within each experiment, 300 to 350 cells were counted for each condition and time point.

### EXAMPLE 10

Inhibition of MLK Proteins Blocks Proliferation and Causes G2 or M-Phase Arrest in HeLa Cells

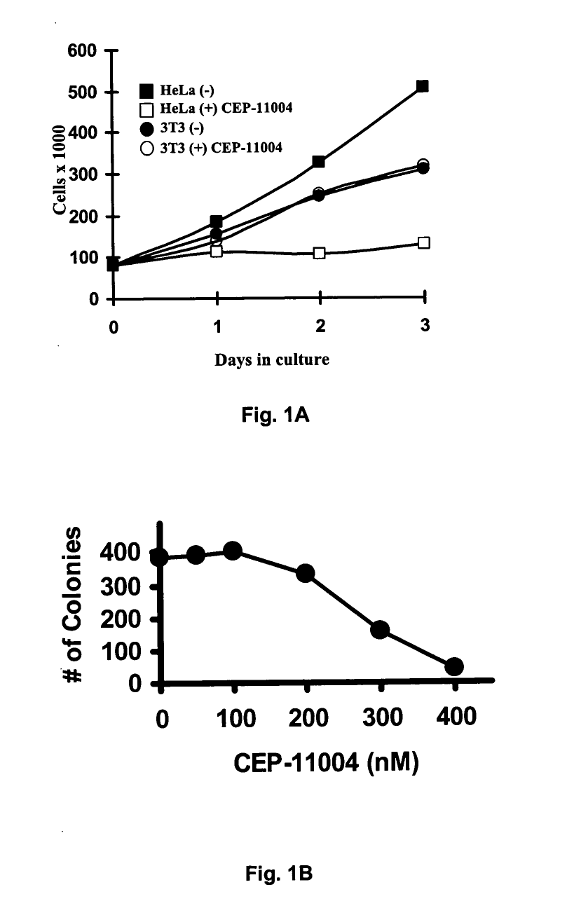

It was recently reported that inhibitors of MLK proteins prevented proliferation of Ras-transformed NIH 3T3 cells, but had no effect on normal NIH 3T3 cells (20). Thus, the effects of MLK inhibition on cell cycle events in transformed cells were analyzed. Whether the addition of the MLK inhibitor CEP-11004 would inhibit proliferation of transformed HeLa cells was tested first. As shown, whereas HeLa cell proliferation was inhibited by CEP-11004, the proliferation of NIH 3T3 cells was unaffected (FIG. 1A). The proliferation of other transformed cells was examined using a colony formation assay. As shown in FIG. 1B, CEP-11004 caused a dose dependent inhibition of A549 airway epithelia carcinoma cell colony formation. A similar inhibition of colony formation was observed in HeLa cells and ER negative SUM159 breast cancer cells treated with CEP-11004 (data not shown).

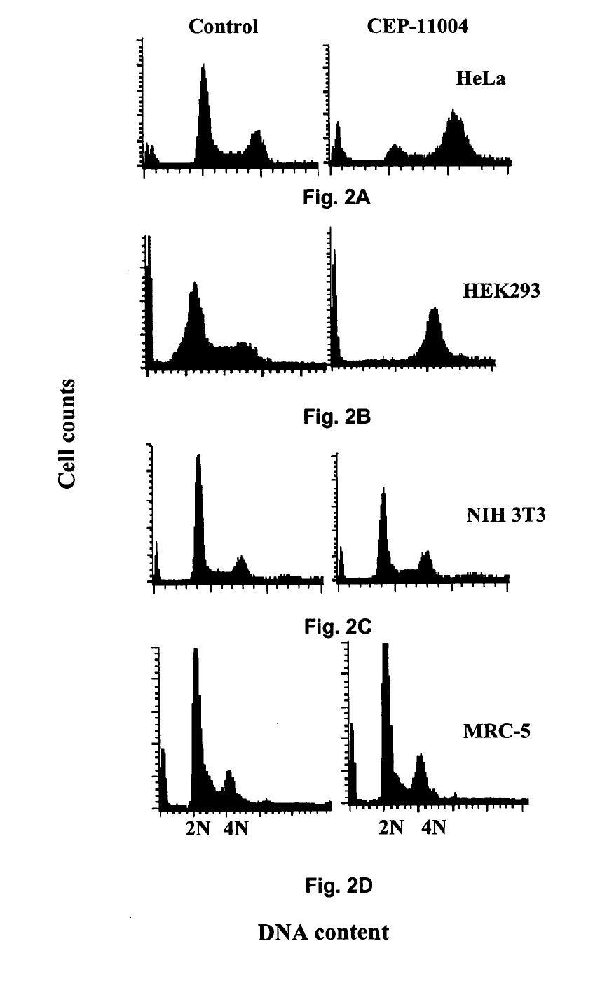

To determine the nature of the CEP-11004 induced inhibition of cell proliferation, FACS analysis of DNA content was used to determine the effects of CEP-11004 on cell cycle progression. As shown, HeLa or HEK293 cells treated with CEP-11004 for 20 hours accumulated 4N DNA indicative of arrest in G2 or M-phase of the cell cycle (FIGS. 2A-2B). In contrast, the DNA content profile in NIH 3T3 cells or normal diploid lung fibroblasts MRC-5 cells was similar in the absence or presence of CEP-11004 (FIGS. 2C-2D).

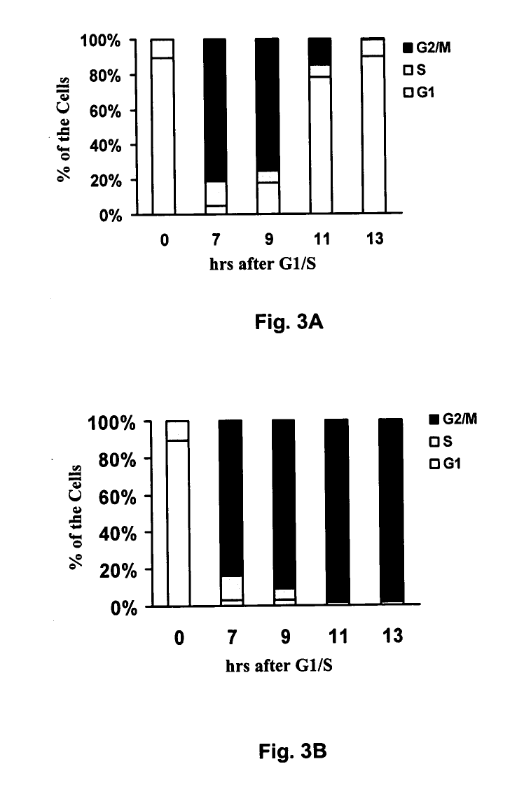

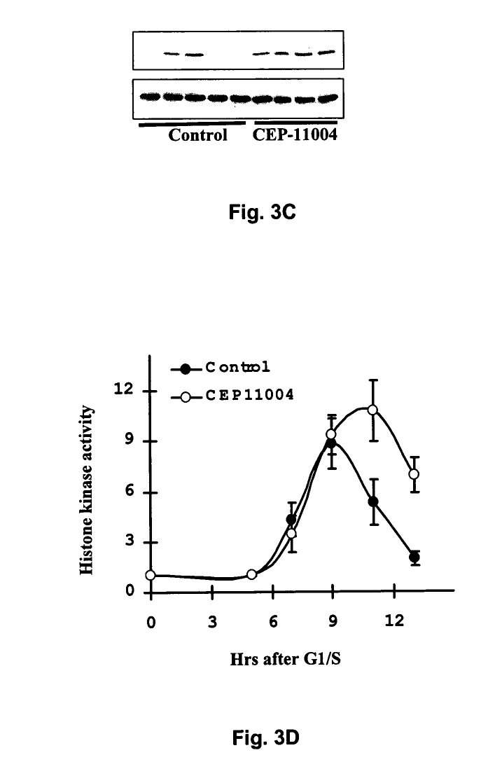

Based on previous data indicating that MLK3 is activated during G2-phase and mitosis (15), the effects of CEP-11004 on cell cycle progression in synchronized HeLa cells was examined. Cells synchronized at the G1/S-phase boundary were released back into the cell cycle for 5 hours to allow completion of most of S-phase. The cells were treated with or without CEP-11004 for varying times and the DNA content was determined by FACS. Between 7 and 9 hours after release from G1/S-block, untreated cells are in G2 and M-phase as demonstrated by the increase in cells with 4N DNA (FIG. 3A). By 11 hours after G1/S release, untreated cells have exited mitosis and re-entered G1-phase (FIG. 3A). In contrast, CEP-11004 treated cells arrest in G2 or M-phase, as evident by the sustained accumulation of cells containing 4N DNA at 11 and 13 hours after G1/S-phase release (FIG. 3B). In agreement, CEP-11004 induced arrest in G2 or M-phase was indicated by the sustained cyclin B1 expression (FIG. 3C) and elevated Cdc2 kinase activity at 11 and 13 hours after G1/S-phase release as compared to untreated synchronized cells (FIG. 3D).

### EXAMPLE 11

CEP-11004 Inhibits MLK3, but not Other Related Kinases

The specificity of CEP-11004 for inhibition of MLK proteins and not other related kinases was examined. These experiments focused on the MLK3 isoform. MLK3 is expressed ubiquitously and is characterized the best of the MLK proteins. Also, although MLK2 shares more than 70% sequence identify in the kinase catalytic domain and other regions with MLK3, MLK2 expression is restricted to brain, skeletal muscle, and testis (21). Additionally, the full sequence of MLK1 has yet to be identified. Finally, MLK3 has recently been implicated to be involved in G2/M-phase transitions (15).

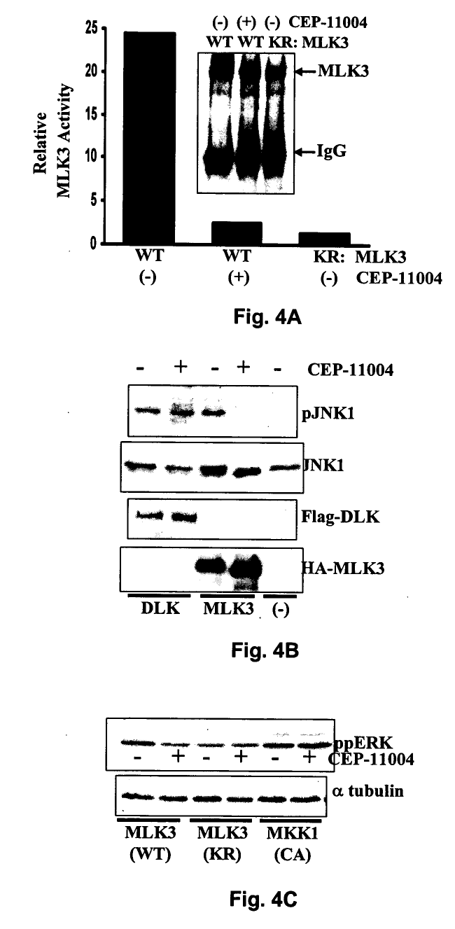

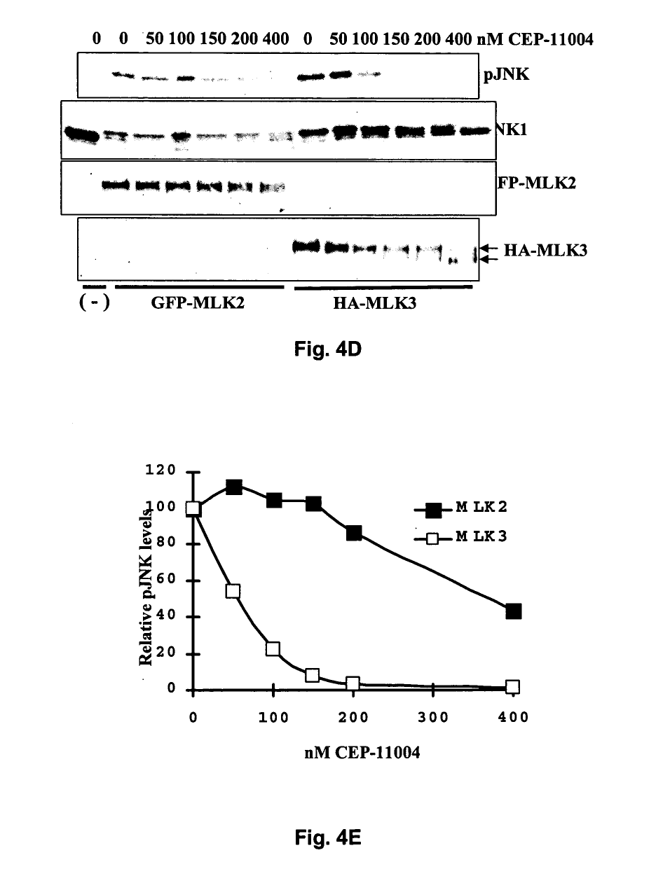

The ability for CEP-11004 to block MLK3 activity was tested first. HA-tagged MLK3 wild type was overexpressed in HeLa cells and treated with or without CEP-11004 for 4 hours before harvesting. As shown in FIG. 4A, CEP-11004 treatment significantly inhibited expressed MLK3 activity compared to untreated cells. Next, the effect of CEP-11004 on a related mixed lineage kinase, dual leucine zipper kinase (DLK), activity was measured in transfected CHO cells. Using JNK MAP kinase phosphorylation as a measure of DLK or MLK3 activity, CEP-11004 inhibited MLK3 but not DLK induced JNK phosphorylation (FIG. 4B). In agreement with others (15), it was demonstrated that MLK3 has higher activity (˜4 fold) during G2 and M-phase transitions as compared to G1/S phase and this activity was blocked by CEP-11004 (data not shown).

Overexpression of MLK3 has been shown to induce ERK activation through MEK1 (13). Therefore, the effect of CEP-11004 on ERK activation following overexpression of MLK3 wild type or a constitutively active MKK1 mutant was examined. Although both MLK3 and active MKK1 stimulate ERK activity, only MLK3-induced ERK activation was inhibited by CEP-11004 (FIG. 4C). Lastly, the ability for CEP-11004 to inhibit MLK2 and MLK3 proteins was compared. GFP-tagged MLK2 or HA-tagged MLK3 were co-expressed with HA-tagged JNK1 for 24 hours and then were treated with or without CEP-11004 for an additional 3 hours before harvesting. Whereas MLK3 induced JNK1 phosphorylation was inhibited by 80% with 100 nM CEP-11004, significant inhibition of MLK2 activation of JNK1 required greater than 400 nM CEP-11004 (FIGS. 4D and E). This is consistent with CEP-11004 having approximately 3 fold higher specificity towards MLK3 as compared to MLK2 (11). Thus, these data support MLK3 as the major MLK isoform targeted by CEP-11004.

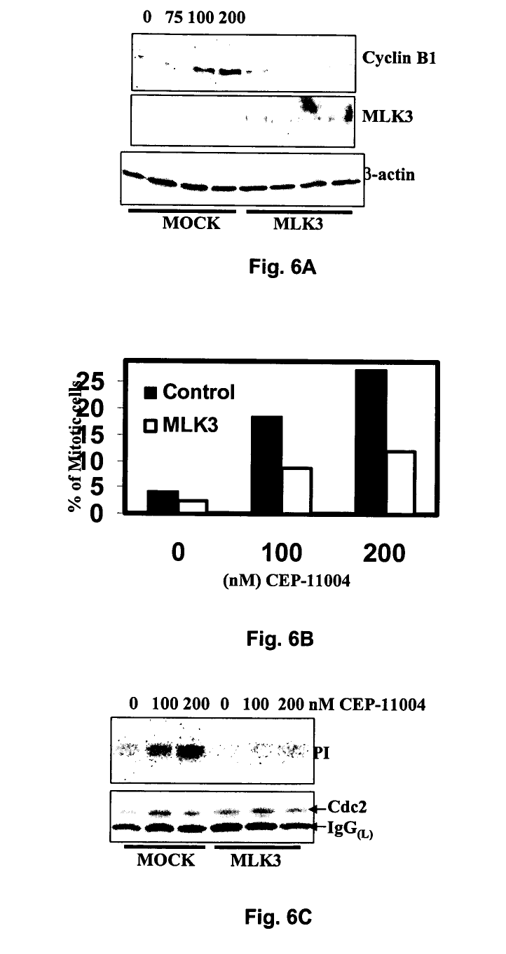

In addition, we demonstrate that CEP-11004 does not affect the activity of other MLK-related (DLK) or non-MLK related protein kinases (MKK1). Nevertheless, others have suggested that the effects of MLK inhibitors on Ras-induced proliferation of NIH 3T3 cells may be due to simultaneous inhibition of the p21-activated kinase-1 (Pak1) (20). Pak1 activity was not tested in the presence or the absence of CEP-11004 because the doses that were used to initiate cell cycle arrest herein were approximately 10 fold less than the reported IC₅₀ concentrations needed for the MLK inhibitor to block Pak1 activity (20). It is contemplated that mitotic arrest occurs at concentrations as low as 100-200 nM (FIGS. 6A-6C).

Another MLK inhibitor, CEP-1347, which has similar biological properties to CEP-11004 was reported to activate the ERK pathway (12). However, these experiments were done in neuronal cells and no evidence that CEP-11004 causes ERK activation in non-neuronal cells has been found. In contrast, as demonstrated, CEP-11004 treatment inhibited MLK3 induced ERK activation (FIG. 4C), which is consistent with a role for MLK3 in activating the ERK pathway in tumor cells (14). This further supports the opposing roles for MLK3 in promoting proliferation of non-neuronal tumor cells but cell death of neuronal cells. In addition, these data suggest a dual function for MLK3 characterized by negative regulation of the ERK pathway in neuronal cells but positive regulation of ERK activity in non-neuronal tumor cells.

### EXAMPLE 12

CEP-11004 Induces Mitotic Arrest in Pro-Metaphase

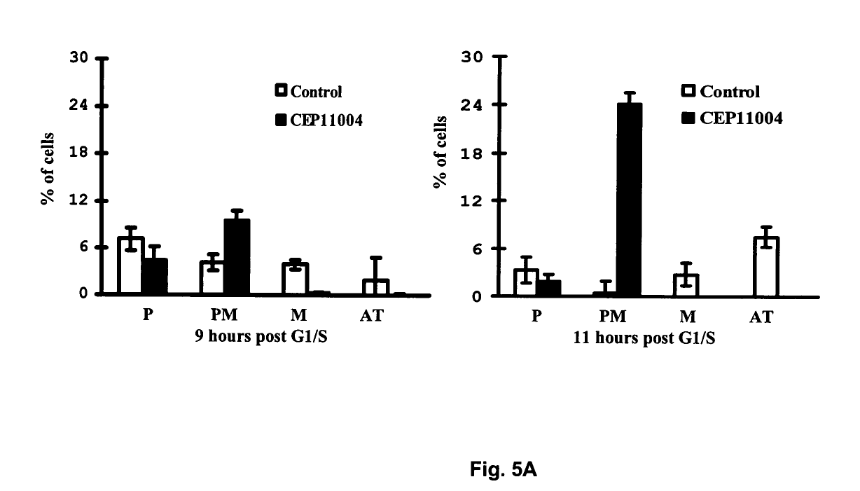

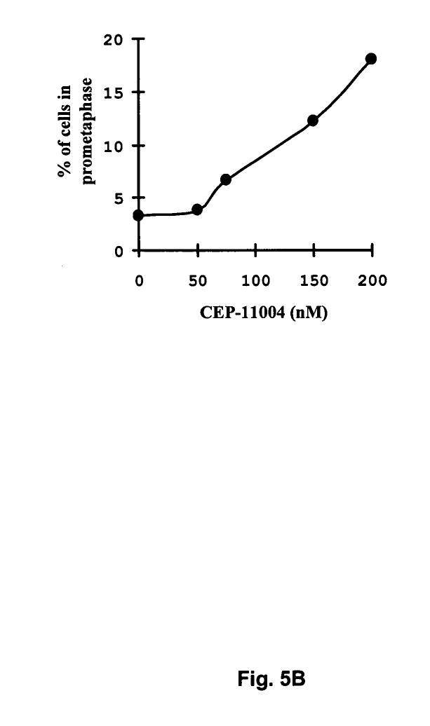

Since it did not appear that CEP-11004 affected progression into mitosis (FIGS. 3A-3D), the nature of CEP-11004 induced M-phase arrest was characterized by examining the various stages of mitosis in synchronized cells treated in the presence or absence of CEP-11004. G1/S-phase cells grown on coverslips were released back into the cell cycle for 5 hours, treated with or without 400 nM CEP-11004 for an additional 4 or 6 hours incubation, i.e., 9 and 11 hours, respectively, after release from G1/S block and the DNA was stained with DAPI. Chromosome morphology was examined by fluorescence microscopy and the percentage of cells in prophase, pro-metaphase, metaphase, or anaphase/telophase was determined. At 9 hours after G1/S, CEP-11004 treated cells began to show accumulation of pro-metaphase cells compared to untreated cells (FIG. 5A, left graph).

Furthermore, cells in metaphase or anaphase/telophase were not evident in CEP-11004 treated cells (FIG. 5A, left graph). This difference became even more apparent at 11 hours after G1/S release as CEP-11004 treated cells primarily accumulated in pro-metaphase unlike control cells, which showed an increased number of anaphase/telophase cells (FIG. 5A, right graph). The number of cells accumulating in pro-metaphase in response to CEP-11004 was dose-dependent. G1/S-phase synchronized cells that were released back into the cell cycle for 5 hours and then treated with various concentrations of CEP-11004 for an additional 8 hours (13 hours after G1/S release), showed a dose-dependent increase in the percentage of pro-metaphase cells (FIG. 5B).

### EXAMPLE 13

MLK3 Overexpression Reverses the Effect of CEP-11004 on Mitotic Arrest

Given that the previous data indicated that inhibition of MLK proteins caused mitotic arrest in pro-metaphase and that MLK3 was a major target of CEP-11004, it was tested whether MLK3 overexpression could reverse mitotic arrest induced by CEP-11004. HeLa cells were co-transfected with pEGFP, to identify transfected cells, in the presence of control vector or HA-MLK3 and then were synchronized at the G1/S boundary using excess thymidine. At 5 hours after G1/S release, cells were incubated with various concentrations of CEP-11004 for an additional 8 hours. Mitotic progression was examined by immunoblotting for cyclin B1, by determining the mitotic index and by assaying Cdc2 kinase activity. Cyclin B1 levels were elevated in mock transfected cells treated with 100 or 200 nM CEP-11004, which is consistent with a mitotic arrest (FIG. 6A). In contrast, MLK3 transfected cells had lower cyclin B1 levels after CEP-1104 treatment consistent with the ability for exogenous MLK3 to prevent the mitotic arrest induced by these concentrations of CEP-11004 (FIG. 6A).

Next, the mitotic index was determined by DAPI staining in mock or MLK3 transfected cells treated with varying concentrations of CEP-11004. As expected, mock transfected cells showed higher levels of mitotic cells following CEP-11004 treatment consistent with mitotic arrest (FIG. 6B). In contrast, MLK3 transfected cells showed a reduced mitotic index following CEP-11004 treatment (FIG. 6B). Lastly, Cdc2 kinase activities in mock or MLK3 transfected cells treated with 100 or 200 nM CEP-11004 were measured. Similar to the cyclin B1 expression, mock transfected cells treated with CEP-11004 exhibited higher Cdc2 activity compared to MLK3 transfected cells (FIG. 6C). These data suggest that exogenous MLK3 is able to overcome the CEP-11004 induced mitotic arrest.

### EXAMPLE 14

CEP-11004 Delays Histone H3 Phosphorylation in Early Mitosis

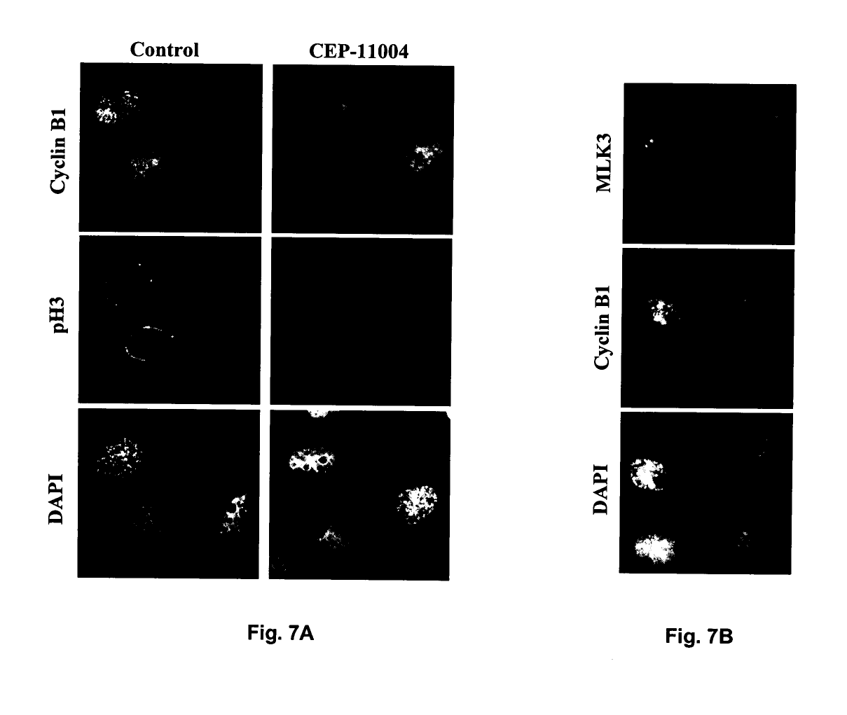

The mechanisms of CEP-11004 induced mitotic arrest were further examined by monitoring mitosis-specific phosphorylation events. Phosphorylation of histone H3 at serine 10 is commonly used as a marker of mitotic progression (22-23). HeLa cells were treated with or without CEP-11004 at 5 hours after release from G1/S-phase thymidine block and allowed to incubate for an additional 4 hours. Cells were then fixed and processed for immunofluorescence of cyclin B1 and phospho-histone H3 (pH3) to identify the mitotic cells. During prophase in control cells, cyclin B1 and pH3 staining were observed in the nucleus prior to nuclear envelope breakdown (FIG. 7A). In contrast, cells treated with CEP-11004 did not contain nuclear pH3 staining during prophase, even though these cells showed strong nuclear reactivity with cyclin B1 (FIG. 7A). This effect on inhibited pH3 staining in early mitotic cells was observed with CEP-11004 doses as low as 200 nM (data not shown). These data suggest that MLK protein activity directly or indirectly is involved in histone H3 phosphorylation during mitosis. This is consistent with the localization of MLK3 in prophase cells, which suggested MLK3 targeting to the nucleus and centrosomes in prophase cells (FIG. 7B).

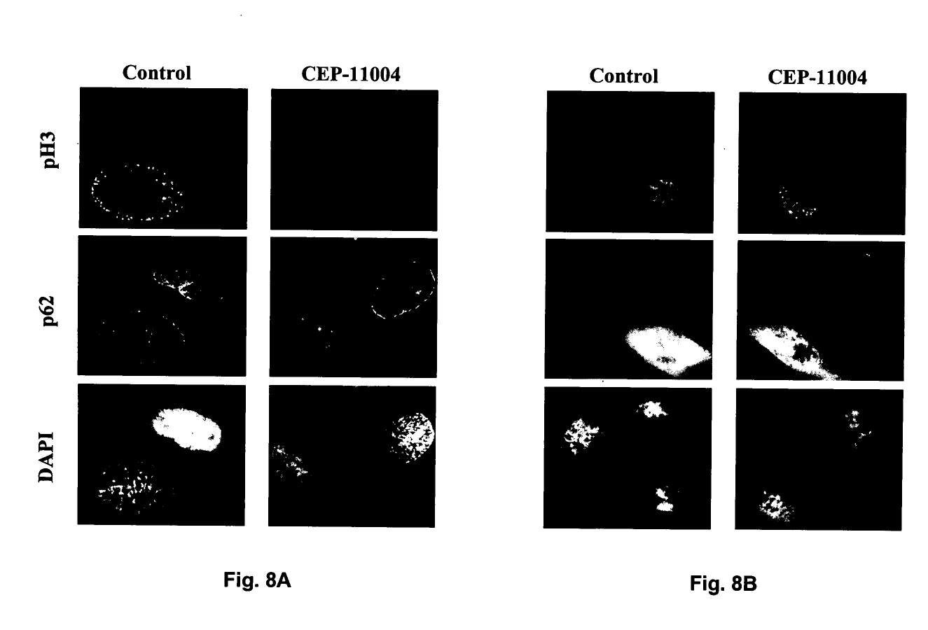

A comparison of pH3 staining before and after nuclear envelope breakdown (NEB) was made. As expected, in untreated cells, pH3 staining was observed in early mitosis before NEB and later stages of mitosis after NEB (FIGS. 8A-8B). The integrity of the nuclear envelope was determined by staining for the p62 nucleoporin protein (FIGS. 8A-8B). Similar to FIGS. 7A-7B, pH3 staining in early mitosis before NEB was not observed in CEP-11004 treated cells (FIG. 8A). However, pH3 staining was readily apparent after NEB in pro-metaphase of both untreated and CEP-11004 treated cells (FIG. 8B). Together, these data indicate that MLK activity is important for histone H3 phosphorylation during early mitosis through a Cdc2/cyclin B1 independent mechanism.

Although CEP-11004 clearly inhibited phosphorylation of histone H3 at S10 in early mitosis, it is not clear whether MLK3 is directly or indirectly responsible for the phosphorylation. Histone H3 is phosphorylated throughout the cell cycle by a number of kinases including PKA, Msk1, ERK, and p38 MAP kinase (27). Although all of these kinases can phosphorylate S10 on histone H3, the functional role of phosphorylation is not clear and may depend on the phase of the cell cycle in which it occurs. Immunofluorescence data suggest that MLK3 might localize to the nucleus of mitotic cells (FIG. 7B) and support a direct phosphorylation of histone H3 by MLK3. Consistent with this is the finding that MLK3 is related to NIMA, a mitotic kinase that phosphorylates S10 on histone H3 in vitro (28).

Even though MLK3 can activate ERK and p38 signaling pathways and histone H3 phosphorylation at serine 10 may be mediated by ERK and p38 MAP kinases in response to stress stimuli (29), it is yet to be determined whether these kinases contribute to histone phosphorylation during early mitosis prior to NEB. Consistent with this is the presence of active ERK in the nucleus of early mitotic cells prior to NEB (30-31). Other potential candidate kinases that regulate phosphorylation of histone H3 at S10 during mitosis are the aurora A and B kinases, with aurora B being the most likely physiological histone H3 kinase since aurora A localizes primarily to the centrosome (32). Nonetheless, aurora B depletion does not eliminate S10 phosphorylation on histone H3 indicating the involvement of other mitotic kinases (27). Although aurora kinases are regulated by phosphorylation, there is no evidence to link MLK proteins with regulation of the aurora kinases.

The significance of the delayed histone H3 phosphorylation observed following inhibition of MLK proteins during mitotic progression is not known. The requirement for phosphorylation of histone H3 on S10 during mitosis remains controversial. For example, although phosphorylation of histone H3 at S10 has been suggested to be required for proper chromosome condensation and mitotic progression in tetrahymena (33-34), a yeast mutant strain containing an alanine mutation at S10 of histone H3 proliferates as well as the wild type strain (35).

### EXAMPLE 15

CEP-11004 Causes Aberrant Spindle Pole Formation During Mitosis

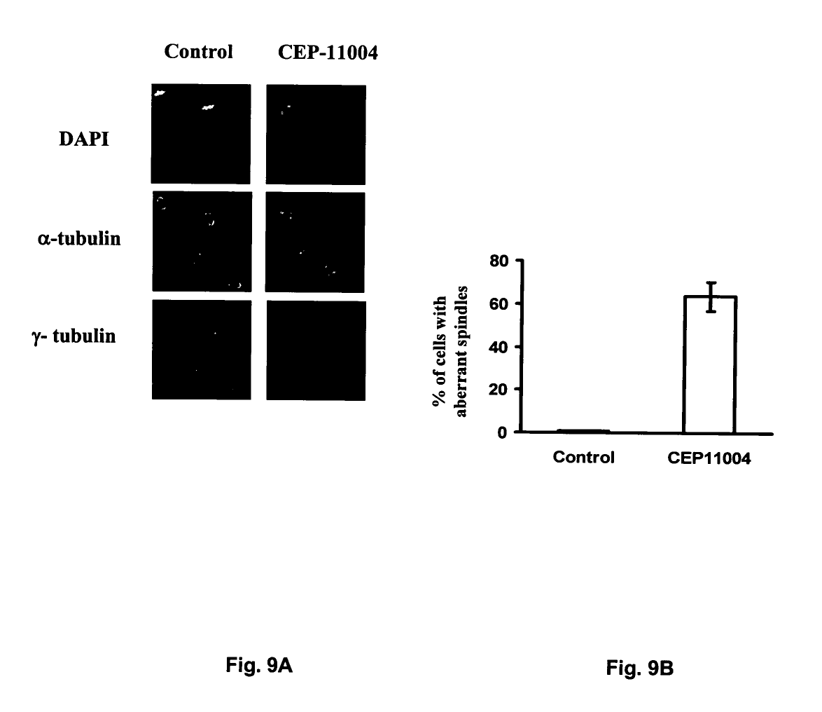

The effects of CEP-11004 on spindle pole assembly in mitotic cells were examined. Synchronized cells were treated at the end of S-phase with or without CEP-11004 for 4 hours followed by immunostaining for α-tubulin as a marker of the microtubules and γ-tubulin as a marker of the spindle pole. As shown, CEP-11004 treatment caused significant disruption of the microtubule organization α-tubulin, FIG. 9A) in mitotic cells, but had no effect on the number of spindle poles (γ-tubulin, FIG. 9A). In addition, the chromosome organization as determined by DAPI staining in CEP-11004 was highly disorganized (FIG. 9A). Greater than 60% of the mitotic cells contained the aberrant microtubule organization (FIG. 9B). Examination of interphase cells treated for 4 hours with CEP-11004 showed no apparent effects on microtubules as compared to untreated cells (data not shown). Thus, the inability for CEP-11004 treated cells to progress through metaphase is likely due to defects in microtubule organization and aberrant spindle formation.

Although overexpression of MLK3 was suggested recently to promote microtubule instability (15), it is not clear what effects inhibition of MLK3 activity has on microtubule organization in mitotic cells. A profound effect on the mitotic microtubule organization within a few hours exposure to CEP-11004 is demonstrated, however, it is unclear what MLK-regulated microtubule associated proteins are being affected. Expression of ectopic MLK3 was shown to destabilize microtubules in HEK293 cells through a mechanism that does not involve JNK or p38 MAP kinase activation (15). However, it is not clear whether this observation is unique to MLK3 or an over-expression artifact. MLK2 and active JNK have also been shown to co-localize along microtubules and interact with motor proteins (24). Whether these interactions were occurring in a cell cycle-dependent manner was not determined. A novel role for direct MLK3 phosphorylation of golgin-160, a Golgi complex protein that may mediate cellular responses to apoptotic stimuli has been described (25). Thus, MLK3 may have additional substrates that may help explain MLK3 functions during the cell cycle that are independent of downstream MAP kinase activation.

It is contemplated that MLK3 inhibition by CEP-11004 prevents cell proliferation by disrupting microtubule events necessary for mitotic progression. This may at first appear to contrast with a previous study that cited unpublished data indicating that down-regulation of MLK3 using RNA interference (RNAi) does not affect cell proliferation or DNA content by flow cytometry (15). However, it is difficult to directly compare the two studies and several reasons could explain the discrepancy between that data (15) and the data herein. First, in previous studies (15), MLK3 was down-regulated in the human osteosarcoma SAOS2 cell line, which reportedly has a functional G2-phase checkpoint, in response to colcemid treatment, as compared to HeLa cells (26). Second, it is unlikely that RNAi treatment is sufficient to completely deplete MLK3 and the remaining MLK3 may be sufficient to promote mitotic progression in these cells. Third, the MLK3-depleted SAOS2 cells were generated by incubation with RNAi for 2-3 days, which could allow other compensatory mechanisms that would substitute for MLK3 during mitotic progression.

The following references were cited herein:
1. Gallo K. A. and Johnson G L. (2002) Nat Rev Mol Cell Biol 3, 663-672.
2. Mata et al. (1996) J Biol Chem 271, 16888-16896.
3. Burbelo et al. (1995) J Biol Chem 270, 290071-29074.
4. Zhang H. and Gallo K. A. (2001) J Biol Chem 276, 45598-45603.
5. Zhang et al. (2004) J Biol Chem 279, 19457-19463.
6. Mota et al. (2001) J Neurosci 21, 4949-4957.
7. Wang et al. (2004) Annu Rev Pharmacol Toxicol 44, 451-474.
8. Xu et al. (2001) Mol Cell Biol 21, 4713-4724.
9. Merritt et al. (1999) J Biol Chem 274, 10195-10202.
10. Tibbles et al. (1996) Embo J 15, 7026-7035.
11. Murakata et al. (2002) Biorg Med Chem Lett 12, 147-150.
12. Roux et al. (2002) J Biol Chem 277, 49473-49480.
13. Hartkamp et al. (1999) Cancer Res 59, 2195-2202.
14. Chadee D. N. and Kyriakis J. M. (2004) Nat Cell Biol 6, 770-776.
15. Swenson et al. (2003) Mol Biol Cell 14, 156-172.
16. Lambert et al. (2002) J Biol Chem 277, 4770-4777.
17. Cho et al. (2004) Cancer Res 64, 3855-3864.
18. Dangi et al. (2003) Cell Signal 15, 667-675.
19. Cha et al. (2004) Biochem J 378, 857-865.
20. Nheu et al. (2002) Cancer J 8, 328-336.
21. Katoh et al. (1995) Oncogene 10, 1447-1451.
22. Hendzel et al. and Allis C. D. (1997) Chromosoma 106, 348-360.
23. Wei et al. (1998) Proc Natl Acad Sci USA 95, 7480-7484.
24. Nagata et al. (1998) Embo J 17, 149-158.
25. Cha et al. (2004) J Cell Sci 117, 751-760.
26. Jha et al. (1994) Cancer Res 54, 5011-5015.
27. Prigent C. and Dimitrov S. (2003) J Cell Sci 116, 3677-3685.
28. De Souza et al. (2000) Cell 102, 293-302.
29. Zhong et al. (2000) J Biol. Chem.
30. Zecevic et al. (1998) J Cell Biol 142, 1547-1558.
31. Shapiro et al. (1998) J Cell Biol 142, 1533-1545.
32. Pascreau et al. (2003) Prog Cell Cycle Res 5, 369-374.
33. Wei et al. (1999) Cell 97, 99-109.
34. Van Hooser et al. (1998) J Cell Sci 111 (Pt 23), 3497-3506.
35. Hsu et al. (2000) Cell 102, 279-291.

Any patents or publications mentioned in this specification are indicative of the levels of those skilled in the art to which the invention pertains. These patents and publications are herein incorporated by reference to the same extent as if each individual publication was specifically and individually incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

1. A method of inhibiting proliferation of a neoplastic cell, comprising:
contacting the neoplastic cell with a compound that selectively inhibits an activity of a mixed-lineage kinase (MLK) in said neoplastic cell, whereby said inhibition of an MLK activity inhibits proliferation of the neoplastic cell.

2. The method of claim 1, wherein said neoplastic cell is a cancer cell.

3. The method of claim 2, wherein said cancer cell is a breast cancer cell, a lung cancer cell, a cervical cancer cell, a pancreatic cancer cell, a bladder cancer cell, a colon cancer cell, or a cancer cell having a Ras mutation.

4. The method of claim 1, wherein said compound is an indolocarbazole molecule.

5. The method of claim 4, wherein said indolocarbazole molecule is CEP-11004 or CEP-1347.

6. The method of claim 1, wherein said mixed lineage kinase is a MLK1 polypeptide comprising SEQ ID NO: 3.

7. The method of claim 1, wherein said mixed lineage kinase is a MLK2 protein comprising SEQ ID NO: 4.

8. The method of claim 1, wherein said mixed lineage kinase is a MLK3 protein comprising SEQ ID NO: 1 or in SEQ ID NO: 2.

9. A method of inhibiting proliferation of a neoplastic cell, comprising:
contacting the neoplastic cell with a compound that selectively inhibits an activity of a mixed lineage kinase (MLK) having a sequence selected from the group consisting of SEQ ID NO:1, SEQ ID NO:2, SEQ ID NO:3, and SEQ ID NO:4,
wherein said inhibition of an MLK activity inhibits proliferation of the neoplastic cell.

10. The method of claim 9, wherein said neoplastic cell is a cancer cell selected from the group consisting of a breast cancer cell, a lung cancer cell, a cervical cancer cell, a pancreatic cancer cell, a bladder cancer cell, a colon cancer cell, and a cancer cell having a Ras mutation.

11. The method of claim 9, wherein said compound is CEP-11004 or CEP-1347.

12. A method of treating a cancer in a subject, comprising:
administering an inhibitor that selectively binds to a mixed-lineage kinase (MLK) or polypeptide thereof in a cancer cell in said subject, wherein said binding inhibits proliferation of said cancer cell, thereby treating the cancer in the subject.

13. The method of claim 12, further comprising:
administering an anticancer drug to the subject.

14. The method of claim 13, wherein said anticancer drug is administered concurrently or sequentially with the inhibitor.

15. The method of claim 13, wherein said anticancer drug is selected from the group consisting of cisplatin, oxaliplatin, carboplatin, doxorubicin, a camptothecin, paclitaxel, methotrexate, vinblastine, etoposide, docetaxel hydroxyurea, celecoxib, fluorouracil, busulfan, imatinib mesylate, alembuzumab, aldesleukin, and cyclophosphamide.

16. The method of claim 13, wherein the dosage of said anticancer drug is lower than a dosage required when said anticancer drug is administered singly, thereby reducing toxicity of the anticancer drug to the individual.

17. The method of claim 12, wherein said cancer is a breast cancer, a lung cancer, a cervical cancer, a pancreatic cancer, a bladder cancer, a colon cancer, or a cancer having a Ras mutation.

18. The method of claim 12, wherein said inhibitor is an indolocarbazole molecule.

19. The method of claim 18, wherein said indolocarbazole molecule is CEP-11004 or CEP-1347.

20. The method of claim 12, wherein said mixed lineage kinase is a MLK1 polypeptide comprising SEQ ID NO: 3.

21. The method of claim 12, wherein said mixed lineage kinase is a MLK2 comprising SEQ ID NO: 4.

22. The method of claim 12, wherein said mixed lineage kinase is a MLK3 comprising SEQ ID NO: 1 or in SEQ ID NO: 2.

23. A method of reducing toxicity of a cancer therapy in an individual in need thereof, comprising:
administering to the individual an inhibitor that selectively binds to a mixed-lineage kinase (MLK) or polypeptide thereof and an anticancer drug, wherein a dosage of the anticancer drug administered with the inhibitor is lower than a dosage required when said anticancer drug is administered singly, thereby reducing toxicity of the cancer therapy to the individual.

24. The method of claim 23, wherein said anticancer drug is administered concurrently or sequentially with the inhibitor.

25. The method of claim 23, wherein said inhibitor is an indolocarbazole molecule.

26. The method of claim 25, wherein said indolocarbazole molecule is CEP-11004 or CEP-1347.

27. The method of claim 23, wherein said mixed lineage kinase is a MLK1 polypeptide comprising SEQ ID NO: 3.

28. The method of claim 23, wherein said mixed lineage kinase is a MLK2 polypeptide comprising SEQ ID NO: 4.

29. The method of claim 23, wherein said mixed lineage kinase is a MLK3 polypeptide comprising SEQ ID NO: 1 or in SEQ ID NO: 2.

30. The method of claim 23, wherein said anticancer compound is selected from the group consisting of cisplatin, oxaliplatin, carboplatin, doxorubicin, a camptothecin, paclitaxel, methotrexate, vinblastine, etoposide, docetaxel hydroxyurea, celecoxib, fluorouracil, busulfan, imatinib mesylate, alembuzumab, aldesleukin, and cyclophosphamide.

31. The method of claim 23, wherein said individual has a cancer selected from the group consisting of a breast cancer, a lung cancer, a cervical cancer, a pancreatic cancer, a bladder cancer, a colon cancer, and a cancer having a Ras mutation.

32. A method of screening for a compound to inhibit mixed lineage kinase (MLK) activity and to arrest proliferation of a neoplastic cell, comprising;
measuring the level of an activity of a MLK protein or of a polypeptide thereof or polypeptide fragment having said MLK activity in the presence or absence of the compound;
comparing the level of MLK activity in the presence of the compound with the level of MLK activity in the absence of the compound, wherein a decrease in MLK activity in the presence of the compound is indicative that the compound has an ability to inhibit MLK activity;
contacting a culture of the neoplastic cells having an activated MLK activity with the compound having an ability to inhibit said MLK activity; and
comparing the amount of cell proliferation of the neoplastic cells in the presence of the inhibitory compound with the amount of cell proliferation of the neoplastic cells in the absence of the inhibitory compound, wherein a decrease in cell proliferation in the presence of the compound compared to cell proliferation in the absence of the compound is indicative that the inhibitory compound has the ability to prevent cell proliferation.

33. The method of claim 32, wherein said mixed lineage kinase is selected from the group consisting of a MLK2 protein, a MLK3 protein, a MLK1 polypeptide, and a polypeptide fragment of MLK1, MLK2 or MLK3 having an MLK activity.

34. The method of claim 33, wherein said MLK1 polypeptide has the sequence shown in SEQ ID NO: 3.

35. The method of claim 33, wherein said MLK2 protein has the sequence shown in SEQ ID NO: 4.

36. The method of claim 33, wherein said MLK3 protein has the sequence shown in SEQ ID NO: 1 or in SEQ ID NO: 2.

37. The method of claim 33, wherein said MLK polypeptide fragment having the MLK activity comprises about 700, 650, 600, 550, 500, 450, 400, 350, 300, 250, 200, 150, or 100 continuous amino acids of the MLK2 or MLK3 proteins or of the MLK1 polypeptide.

38. The method of claim 33, wherein said neoplastic cell is a cancer cell.

39. The method of claim 38, wherein said cancer cell is a breast cancer cell, a lung cancer cell, a cervical cancer cell, a pancreatic cancer cell, a bladder cancer cell, a colon cancer cell, or a cancer cell having a Ras mutation.

40. A compound identified by the method of claim 32.


SEQUENCE LISTING (4 sequences)

SEQ ID NO: 1 - protein, 847 aa, Homo sapiens
  MISC_FEATURE (1)..(847): Amino acid sequence for Human MLK3
MEPLKSLFLKSPLGSWNGSGSGGGGGGGGGRPEGSPKAAGYANPVWTALFDYEPSGQDELALRKGDRVEVLSRDAAISGDEGWWAGQVGGQVGIFPSNYVSRGGGPPPCEVASFQELRLEEVIGIGGFGKVYRGSWRGELVAVKAARQDPDEDISVTAESVRQEARLFAMLAHPNIIALKAVCLEEPNLCLVMEYAAGGPLSRALAGRRVPPHVLVNWAVQIARGMHYLHCEALVPVIHRDLKSNNILLLQPIESDDMEHKTLKITDFGLAREWHKTTQMSAAGTYAWMAPEVIKASTFSKGSDVWSFGVLLWELLTGEVPYRGIDCLAVAYGVAVNKLTLPIPSTCPEPFAQLMADCWAQDPHRRPDFASILQQLEALEAQVLREMPRDSFHSMQEGWKREIQGLFDELRAKEKELLSREEELTRAAREQRSQAEQLRRREHLLAQWELEVFERELTLLLQQVDRERPHVRRRRGTFKRSKLRARDGGERISMPLDFKHRITVQASPGLDRRRNVFEVGPGDSPTFPRFRAIQLEPAEPGQAWGRQSPRRLEDSSNGERRACWAWGPSSPKPGEAQNGRRRSRMDEATWYLDSDDSSPLGSPSTPPALNGNPPRPSLEPEEPKRPVPAERGSSSGTPKLIQRALLRGTALLASLGLGRDLQPPGGPGRERGESPTTPPTPTPAPCPTEPPPSPLICFSLKTPDSPPTPAPLLLDLGIPVGQRSAKSPRREEEPRGGTVSPPPGTSRSAPGTPGTPRSPPLGLISRPRPSPLRSRIDPWSFVSAGPRPSPLPSPQPAPRRAPWTLFPDSDPFWDSPPANPFQGGPQDCRAQTKDMGAQAPWVPEAGP

SEQ ID NO: 2 - protein, 850 aa, Mus musculus
  MISC_FEATURE (1)..(850): Amino acid sequence for mouse MLK3
MEPLKNLFLKSPLGSWNGSGSGGGGGTGGVRPEGSPKATAAYANPVWTALFDYEPNGQDELALRKGDRVEVLSRDAAISGDEGWWAGQVGGQVGIFPSNYVSRGGGPPPCEVASFQELRLEEVIGIGGFGKVYRGSWRGELVAVKAARQDPDEDISVTAESVRQEARLFAMLAHPNIIALKAVCLEEPNLCLVMEYAAGGPLSRALAGRRVPPHVLVNWAVQIARGMHYLHCEALVPVIHRDLKSNNILLLQPIEGDDMEHKTLKITDFGLAREWHKTTQMSAAGTYAWMAPEVIKASTFSKGSDVWSFGVLLWELLTGEVPYRGIDCLAVAYGVAVNKLTLPIPSTCPEPFAQLMADCWAQDPHRRPDFASILQQLEALEAQVLREMPRDSFHSMQEGWKREIQGLFDELRAKEKELLSREEELTRAAREQRSQAEQLRRREHLLAQWELEVFERDVTLLLQQVDRERPHVRRRRGTFKRSKLRARDGGERISMPLDFKHRITVQASPGLDRRRNVFEVGAGDSPTFPRFRAIQLEPTESGQTWGRQSPRRLEDSSNGERRACWAWGPSSPKPGEAQNGRRRSRMDEATWYLDSDDSSPLGSPSTPPALNGNPPRPSPEPEEPRRAGPTERGNSSGTPKLIQRALLRGTALLASLGLGRDLQPPGGLSRERGESPTAPPPAQMPSPCPPELPSTPLIRLSQTTPDAHSSPTPGPLLLDLGVPSGQPSAKSPRREETRGRTVSPPPGISRSAPGTPGTPRSPPLGLISRPRPSPLRSRIDPWSFVSAGPRPSPLPSPQPAPRRAPWTLFPDSDPFWDSPPANPFRGGSQDCRTQTKDMGCPAPWAPEAGP

SEQ ID NO: 3 - protein, 394 aa, Homo sapiens [sequence omitted]

SEQ ID NO: 4 - protein, 954 aa, Homo sapiens [sequence omitted]